(19) United States
(12) Patent Application Publication
(10) Pub. No.: US 2001/0000524 A1
(43) Pub. Date: Apr. 26, 2001

(54) Carboxylated polymers complexes

(76) Inventors: Anthony J. O'Lenick JR., Dacula, GA (US); Charles W. Buffa, Paterson, NJ (US)

Correspondence Address: A.J. O'Lenick, Jr., 2170 Luke Edwards Road, Dacula, GA, 30019, US

(21) Appl. No.: 09/734,828

(22) Filed: Dec. 13, 2000

Related U.S. Application Data
(62) Division of application No. 09/425,896, filed on Oct. 25, 1999.

(51) Int. Cl. C07C 69/76; C07C 69/34; C07C 69/52

(52) U.S. Cl. 560/199; 560/112; 564/281

(57) ABSTRACT

The present invention deals with the composition, and application of novel carboxylated polymers. The complexes are made by a series of reactions which include reaction of an anhydride with an allyl alcohol alkoxylate, polymerization of the carboxylated allyl alkoxylate, then formation of a complex of the poly carboxylate with quaternary compounds.

### RELATED APPLICATION

1. This application is a divisional patent application of co-pending Ser. No. 09/425,896 filed Oct. 25, 1999.

### BACKGROUND OF THE INVENTION

2. 1. Field of the Invention

3. The present invention deals with the composition, and application of novel carboxylated polymers, and complexes thereof. The complexes useful as softening, anti-tangle, and conditioning agents for use in personal care applications due to their outstanding mildness. The properties of these novel compounds that makes them well suited for these applications is the fact that they are substantive to fibers, hair and skin and also very mild to the skin and eyes and provide protection from environmental factors like acid rain and other pollutions which come in contact with hair and skin. The use of the complex results in several additional desirable properties heretofore unattainable. This includes overcoming the problem of eye and skin irritation using traditional cationic conditioning agents.

4. 2. Arts and Practices

5. U.S. Pat. No. 5,162,472 to O'Lenick discloses free radical polymers which incorporate silicone into the backbone. While these materials have desirable properties, they do not form monolayers on the hair nor do they provide protection to the hair.

6. The formation of a high molecular weight polymeric complex results in less penetration of the skin by the compounds of the present invention and a very effective conditioner that remains on the surfact of the hair or skin where the conditioning agent is most effective.

7. The references cited herein are incorporated by reference to the extent applicable. Ratios and percentages are by weight and temperatures are Celsius unless otherwise stated.

### THE INVENTION

### Object of the Invention

8. It is the object of the current invention to provide a novel series of anionic free radical polymers and complexes thereof with cationic compounds. The formation of a high molecular weight polymeric complex results in less penetration of the skin by the compounds of the present invention and a very effective conditioner that remains on the surfact of the hair or skin where the conditioning agent is most effective.

9. It is another object of the current invention to provide a novel allyl alkoxy carboxylate used as an intermediate in the preparation of the compounds of the present invention.

10. It is still another objective of the current invention to provide personal care compositions which contain an effective conditioning amount of the compounds of the current invention. That effective conditioning concentration will vary from 0.1 to 20% of the composition. The compounds of the present invention have outstanding conditioning properties when applied to hair and skin.

### SUMMARY OF THE INVENTION

11. The present invention is directed to free radical polymers which contain a carboxyl group as one of the functional groups polymerized. The compounds of the invention are prepared by the free radical polymerization of a novel allyl carboxy monomer.

12. The carboxy functional allyl monomer is polymerized and subsequently complexed with a variety of quaternary compounds to form the conditioning quats of the present invention.

13. The allyl alkoxy carboxylate used to make the compounds of the present invention conform to the following structure:

CH₂═CH—CH₂—O—(CH₂CH₂O)ₐ—(CH₂CH(CH₃)O)_{b}—(CH₂CH₂O)_{c}—R¹

14. wherein:

15. a, b and c are integers each independently ranging from 0 to 20;

16. R¹ is selected from the group consisting of;

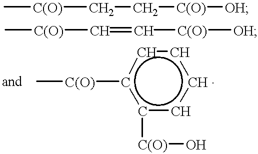

17. The compounds are made by the reaction of allyl alcohol alkoxylates, which are commercially available with succinic anhydride, maleic anhydride and phthalic anhydride.

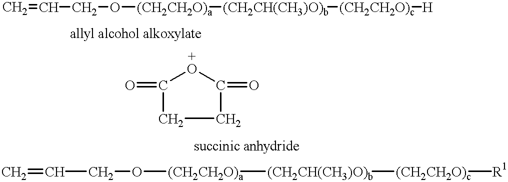

18. wherein R¹ is —C(O)—CH₂—CH₂—C(O)—OH.

19. The same reaction is conducted with maleic anhydride and phthalic anhydride to give the other compounds.

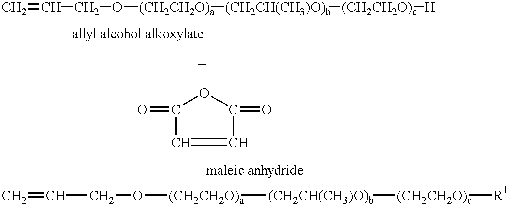

20. wherein R¹ is —C(O)—CH₂═CH₂—C(O)—OH.

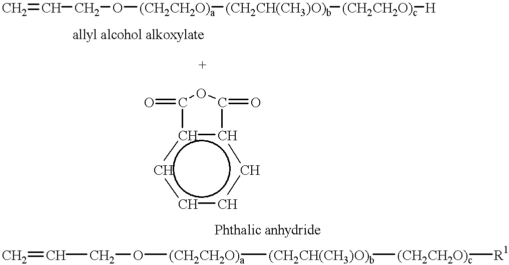

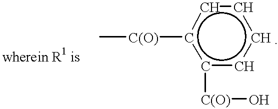

21. These allyl alkoxy carboxylates are key materials to synthesise the carboxy polymers that are used to make the ultimate complex. The carboxy polymers conform to the following structure;

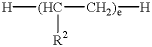

22. wherein;

23. R² is CH₂CH₂—CH₂—O—(CH₂CH₂O)ₐ—(CH₂CH(CH₃)O)_{b}—(CH₂CH₂O)_{c}—R¹

24. e is an integer from 10 to 2,000.

25. The compounds of the current invention are prepared by the free radical reaction of the allyl alkoxy carboxylate containing monomer.

### COMPLEX

26. The complex compound of the present invention conform to the following structure;

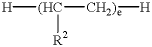

27. wherein;

28. R² is CH₂CH₂—CH₂—O—(CH₂CH₂O)ₐ—(CH₂CH(CH₃)O)_{b}—(CH₂CH₂O)_{c}—R³

29. R³ is selected from the group consisting of;

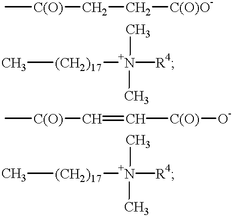

30. and

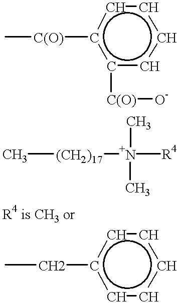

31. e is an integer from 10 to 2,000.

### PREFERRED EMBODIMENTS

32. In a preferred embodiment of the allyl alkoxy carboxylate R¹ is —C(O)—CH₂—CH₂—C(O)—OH.

33. In a preferred embodiment of the allyl alkoxy carboxylate R¹ is —C(O)—CH═CH—C(O)—OH.

34. In a preferred embodiment of the allyl alkoxy carboxylate

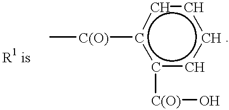

35. In a preferred embodiment of the allyl alkoxy carboxylate x, y, and z are each 0.

36. In a preferred embodiment of the allyl alkoxy carboxylate x ranges from 7 to 15 y is 0 and z is 0.

37. In a preferred embodiment of the carboxy polymer R¹ is —C(O)—CH₂—CH₂—C(O)—OH.

38. In a preferred embodiment of the carboxy polymer R¹ is —C(O)—CH═CH—C(O)—OH.

39. In a preferred embodiment of the carboxy polymer

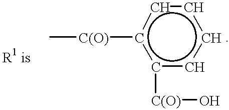

40. In a preferred embodiment of the carboxy polymer x, y, and z are each 0.

41. In a preferred embodiment of the carboxy polymer x ranges from 7 to 15 y is 0 and z is 0.

42. In a preferred embodiment of the complex R³ is

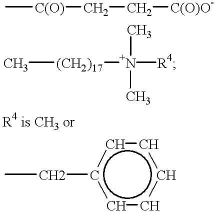

43. In a preferred embodiment of the complex R³ is

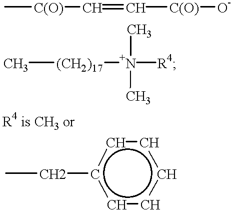

44. In a preferred embodiment of the complex R³ is

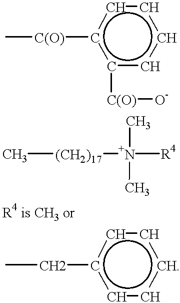

45. In a preferred embodiment of the complex R⁴ is CH₃.

46. In a preferred embodiment of the complex R⁴ is CH₃.

### EXAMPLES

47.

Raw Materials
These compounds conform to the following structure:
CH₂═CH—CH₂—O—(CH₂CH₂—O)ₓ—(CH₂CH(CH₃)CH₂—O)_{y}—(CH₂CH₂—O)_{z}—H

| Example Number | x | y | z |
|---|---|---|---|
| 1 | 0 | 0 | 0 |
| 2 | 0 | 1 | 0 |
| 3 | 4 | 0 | 0 |
| 4 | 7 | 0 | 0 |
| 5 | 10 | 0 | 0 |
| 6 | 20 | 0 | 0 |
| 7 | 4 | 7 | 10 |
| 8 | 7 | 4 | 20 |
| 9 | 10 | 20 | 4 |
| 10 | 20 | 10 | 7 |
| 11 | 20 | 20 | 20 |
| 12 | 7 | 7 | 7 |

48. These materials are items of commerce available commercially from Siltech Corporation Toronto Ontario Canada, and Pelron Corporation Chicago Ill.

### Preparation of Allyl Alkoxy Carboxylates

49. General Procedure

50. Carboxy allyl alkoxylates are prepared by the reaction of the allyl alcohol alkoxylates example 1-12 with one mole of anhydride. The reaction mass is heated to 80 to 100° C. and the theoretical amount of water is stripped off.

### Succinic Anhydride Examples

51. To 103.0 grams of succinic anhydride is added to a clean glass vessel equipped with agitation and a thermometer. Next, the specified amount of the specified allyl alkoxylate Examples 1-12 is added. The reaction mass is heated to 80 to 100 C. and the theoretical amount of water distills off. The allyl carboxylic ester is used without additional purification.

### Examples 13-32

52.

| | Allyl Alkoxylate | |
|---|---|---|
| | Grams | Example Number |
| Example 13 | 58.0 | 1 |
| Example 14 | 102.0 | 2 |
| Example 15 | 234.0 | 3 |
| | Allyl alkoxylate | Allyl alkoxylate |
| | Grams | Example Number |
| Example 16 | 366.0 | 4 |
| Example 17 | 498.0 | 5 |
| Example 18 | 938.0 | 6 |
| Example 19 | 1087.0 | 7 |
| Example 20 | 1718.0 | 8 |
| Example 21 | 1912.1 | 9 |
| Example 22 | 1836.2 | 10 |
| Example 23 | 2998.3 | 11 |
| Example 24 | 1087.0 | 12 |

### Maleic Anhydride Examples

53. To 101.0 grams of maleic anhydride is added to a clean glass vessel equipped with agitation and a thermometer. Next, the specified amount of the specified intermediate Examples 1-12 is added. The reaction mass is heated to 80 to 100 C. and the theoretical amount of water distills off. The allyl carboxylic ester is used without additional purification.

### Examples 25-36

54.

| | Allyl Alkoxylate | Allyl Alkoxylate |
|---|---|---|
| | Grams | Example Number |
| Example 25 | 58.0 | 1 |
| Example 26 | 102.0 | 2 |
| Example 27 | 234.0 | 3 |
| Example 28 | 366.0 | 4 |
| Example 29 | 498.0 | 5 |
| Example 30 | 938.0 | 6 |
| Example 31 | 1087.0 | 7 |
| Example 32 | 1718.0 | 8 |
| Example 33 | 1912.1 | 9 |
| Example 34 | 1836.2 | 10 |
| Example 35 | 2998.3 | 11 |
| Example 36 | 1087.0 | 12 |

### Examples 37-48

### Phthalic Anhydride Examples

55. To 146.0 grams of phthalic anhydride is added to a clean glass vessel equipped with agitation and a thermometer. Next, the specified amount of the specified intermediate Examples 1-12 is added. The reaction mass is heated to 80 to 100 C. and the theoretical amount of water distills off. The allyl carboxylic ester is used without additional purification.

| | Allyl alkoxylate | Allyl alkoxylate |
|---|---|---|
| | Grams | Example Number |
| Example 37 | 58.0 | 1 |
| Example 38 | 102.0 | 2 |
| Example 39 | 234.0 | 3 |
| Example 40 | 366.0 | 4 |
| Example 41 | 498.0 | 5 |
| Example 42 | 938.0 | 6 |
| Example 43 | 1087.0 | 7 |
| Example 44 | 1718.0 | 8 |
| Example 45 | 1912.1 | 9 |
| Example 46 | 1836.2 | 10 |
| Example 47 | 2998.3 | 11 |
| Example 48 | 1087.0 | 12 |

### Preparation of Carboxy Polymers

### Examples 49-84

56. General Polymerization Procedure

57. The polymerization of the allyl carboxy compound is achieved by utilizing free radical catalyst in a low oxygen containing solvent, most commonly water. The water is deionized and sparged with nitrogen to remove dissolved oxygen contained therein immediately prior to use. Then, the specified amount of the treated de-ionized water is added to a suitable glass vessel. Most commonly, 50 to 80% of the total weight of the batch is water. The specified amount of the specified monomers are then added under agitation. Nitrogen is continuously sparged and the temperature is raised to about 50 C. Once the temperature has reached 50 and the nitrogen has been bubbled through the reaction mass for thirty minutes, a free radical initiator is added. Many peracids, like t-butyl-perbenzoate, t-butyl-hydroperoxide and inorganic free radical initiators like stannic chloride can be used. The preferred initiator is azobisisobutylnitrile. The reaction is exothermic and cooling is used to keep the temperature below 90 C.

58. The molecular weight is monitored by viscosity and both increase as the reaction continues.

### Example 49

59. To the 5,000 grams of deionized water, which has just been spargred with nitrogen for 30 minutes, is added the specified amount 5,000 grams of the specified allyl carboxy monomer Ex # 13 under good agitation and nitrogen sparge. The temperature is raised to about 50 C. Once the temperature has reached 50 and the nitrogen has been bubbled through the reaction mass for thirty minutes, 0.05% by weight of batch of azobisisobutylnitrile. The catalyst may be optimally added in smaller increments of one quarter of the total needed waiting 30 minutes between additions. The viscosity will raise as the polymerization occurs. The temperature raises to about 90 C. and is cooled with cooling water as needed to prevent the temperature from reaching 90 C. The molecular weight is controlled by viscosity. When the desired visocity is achieved, air is bubbled through the vessel to quench the polymer. The desired polymer is used as prepared.

### Examples 50-84

60. Example 49 is repeated only substituting the allyl carboxy monomer for example 13 used in example 49.

| | | Final Viscosity |
|---|---|---|
| Example | Allyl Carboxy Example | (centipose) |
| 50 | 14 | 100 |
| 51 | 15 | 500 |
| 52 | 16 | 1000 |
| 53 | 17 | 2000 |
| 54 | 18 | 500 |
| 55 | 19 | 700 |
| 56 | 20 | 1200 |
| 57 | 21 | 5000 |
| 58 | 22 | 500 |
| 59 | 23 | 50 |
| 60 | 24 | 1250 |
| 61 | 25 | 1375 |
| 62 | 26 | 5000 |
| 63 | 27 | 8250 |
| 64 | 28 | 1000 |
| 65 | 29 | 500 |
| 66 | 30 | 800 |
| 67 | 31 | 2500 |
| 68 | 32 | 5000 |
| 69 | 33 | 7000 |
| 70 | 34 | 9000 |
| 71 | 35 | 100 |
| 72 | 36 | 500 |
| 73 | 37 | 900 |
| 74 | 38 | 1250 |
| 75 | 39 | 1450 |
| 76 | 40 | 780 |
| 77 | 41 | 6000 |
| 78 | 42 | 8000 |
| 79 | 43 | 7500 |
| 80 | 44 | 1500 |
| 81 | 45 | 280 |
| 82 | 46 | 1350 |
| 83 | 47 | 4000 |
| 84 | 48 | 1000 |

### Polymeric Complexes

61. Cationic Component

### Stearalkonium Chloride

62. Stearalkonium Chloride is an item of commerce available from a variety of sources. One such source is Henkel Corporation in Hoboken N.J..

63. Stearylalkonium chloride is also known as n,n-dimethyl, n-stearyl, n benzyl ammonium chloride and conforms to the following structure:

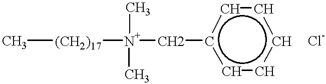

64. Stearyl trimethyl ammonium chloride

65. Stearyl trimethyl ammonium chloride is an item of commerce available from a variety of sources. One such source is Henkel Corporation in Hoboken N.J..

66. Stearyl trimethyl ammonium chloride is also known as n,n, n-trimethyl,n-stearyl ammonium chloride and conforms to the following structure:

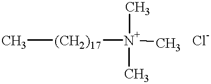

### Preparation of Complex

### Example 85

67. The polymers of the present invention (examples 51-84) are typically prepared solutions ranging in concentrations of between 10 and 55% solids. The examples given use a 50% solid product.

68. To 410.0 grams of the 50% solution of polymer example 50 is added 500 grams of water. The resulting solution is heated to 80 C. In a separate container the quaternary compound is heated to 80 C. The molten quat is added to the hot polymer solution under good agitation. The pH is adjusted to 7.0 using NaOH 50%. The result is a clear homogenous complex of quaternary and carboxy polymer.

### Examples 86-119

69. Example 85 is repeated, only this time the specified number of grams of the specified polymer example replaces the polymer used in example 85. 500 grams of water is added. The resulting solution is heated to 80 C. In a separate container the quaternary compound is heated to 80 C. The molten quat is added to the hot polymer solution under good agitation. The pH is adjusted to 7.0 using NaOH 50%. The result is a clear homogenous complex of quaternary and carboxy polymer.

| Example | Grams | Example | type | Grams |
|---|---|---|---|---|
| 86 | 674.0 | 51 | stearalkonium | 424.0 |
| | | | chloride | |
| 87 | 572.0 | 52 | stearalkonium | 424.0 |
| | | | chloride | |
| 88 | 704.0 | 53 | stearalkonium | 424.0 |
| | | | chloride | |
| 89 | 2082.0 | 54 | stearalkonium | 424.0 |
| | | | chloride | |
| 90 | 1293.0 | 55 | stearalkonium | 424.0 |
| | | | chloride | |
| 91 | 3642.0 | 56 | stearalkonium | 424.0 |
| | | | chloride | |
| 92 | 2118.0 | 57 | stearalkonium | 424.0 |
| | | | chloride | |
| 93 | 3878.0 | 58 | stearalkonium | 424.0 |
| | | | chloride | |
| 94 | 6202.6 | 59 | stearalkonium | 424.0 |
| | | | chloride | |
| 95 | 2380.0 | 60 | stearalkonium | 424.0 |
| | | | chloride | |
| 96 | 318.0 | 61 | stearalkonium | 424.0 |
| | | | chloride | |
| 97 | 406.0 | 62 | stearalkonium | 424.0 |
| | | | chloride | |
| 98 | 670.0 | 63 | stearalkonium | 424.0 |
| | | | chloride | |
| 99 | 934.0 | 64 | stearalkonium | 424.0 |
| | | | chloride | |
| 100 | 1198.0 | 65 | stearalkonium | 424.0 |
| | | | chloride | |
| 101 | 2978.0 | 66 | stearalkonium | 424.0 |
| | | | chloride | |
| 102 | 2376.0 | 67 | stearyltrimethyl | 348.0 |
| | | | ammonium chloride | |
| 103 | 1920.0 | 68 | stearyltrimethyl | 348.0 |
| | | | ammonium chloride | |
| 104 | 4026.0 | 69 | stearyltrimethyl | 348.0 |
| | | | ammonium chloride | |
| 105 | 3874.0 | 70 | stearyltrimethyl | 348.0 |
| | | | ammonium chloride | |
| 106 | 6198.0 | 71 | stearyltrimethyl | 348.0 |
| | | | ammonium chloride | |
| 107 | 2466.0 | 72 | stearyltrimethyl | 348.0 |
| | | | ammonium chloride | |
| 108 | 408.0 | 73 | stearyltrimethyl | 348.0 |
| | | | ammonium chloride | |
| 109 | 496.0 | 74 | stearyltrimethyl | 348.0 |
| | | | ammonium chloride | |
| 110 | 760.0 | 75 | stearyltrimethyl | 348.0 |
| | | | ammonium chloride | |
| 111 | 1024.0 | 76 | stearyltrimethyl | 348.0 |
| | | | ammonium chloride | |
| 112 | 1288.0 | 77 | stearyltrimethyl | 348.0 |
| | | | ammonium chloride | |
| 113 | 2168.0 | 78 | stearyltrimethyl | 348.0 |
| | | | ammonium chloride | |
| 114 | 2466.0 | 79 | stearyltrimethyl | 348.0 |
| | | | ammonium chloride | |
| 115 | 3728.0 | 80 | stearyltrimethyl | 348.0 |
| | | | ammonium chloride | |
| 116 | 4116.0 | 81 | stearyltrimethyl | 348.0 |
| | | | ammonium chloride | |
| 117 | 3965.0 | 82 | stearyltrimethyl | 348.0 |
| | | | ammonium chloride | |
| 118 | 3144.3 | 83 | stearyltrimethyl | 348.0 |
| | | | ammonium chloride | |
| 119 | 1233.0 | 84 | stearyltrimethyl | 348.0 |
| | | | ammonium chloride | |

### Applications Examples

70. The polymers of the present invention are very substantive conditioners to the hair and are surprisingly mild to the skin and eyes. Eye irritation is a major concern in the formulation of personal care products, particularly when working with quats. Primary eye irritation was tested using the protocol outlined in FHSLA 16 CFR 1500.42. The products were tested at 25% actives. The results were as follows:

| Compound | Score | Description |
|---|---|---|
| Stearyl trimethyl | 106.0 | Severely Irritating |
| ammonium chloride | | |
| Example 102 | 8.3 | Minimally Irritating |
| Example 115 | 6.2 | Minimally Irritating |
| Stearalkonium Chloride | 116.5 | Severely Irritating |
| Example 87 | 11.3 | Minimally |
| | | Irritating |
| Example 89 | 6.0 | Minimally |
| | | Irritating |

71. As the data clearly shows, the irritation potential of the complex is dramatically reduced, when compared to the starting quat.

What is claimed:

1. A complex conforming to the following structure: 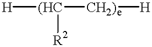
wherein;
R² is CH₂CH₂—CH₂—O—(CH₂CH₂O)ₐ—(CH₂CH(CH₃)O)_{b}—(CH₂CH₂O)_{c}—R³
R³ is selected from the group consisting of; 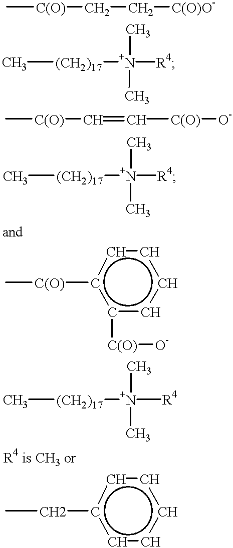
e is an integer from 10 to 2,000.

2. A complex of claim 1 wherein R³ is

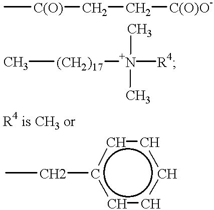

3. A complex of claim 1 wherein R³ is

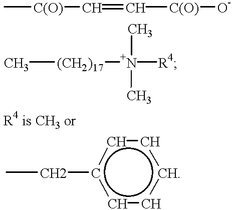

4. A complex of claim 1 wherein R³ is

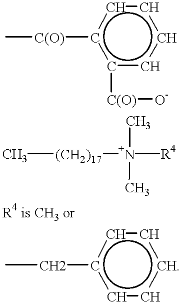

5. A complex of claim 2 wherein R⁴ is CH₃.

6. A complex of claim 3 wherein R⁴ is CH₃.

7. A complex of claim 4 wherein R⁴ is CH₃.

8. A complex of claim 1 wherein R⁴ is [chemical structure]
